(19) United States
(12) Patent Application Publication
(10) Pub. No.: US 2012/0152764 A1
(43) Pub. Date: Jun. 21, 2012

(54) HEMOGLOBIN-DETECTING ELECTRODE TEST STRIP AND DEVICE COMPRISING THE SAME

(75) Inventors: Yueh-Hui LIN, Hsinchu (TW); Yi-Feng Cheng, Hsinchu (TW); Chih-Hsuan Chang, Hsinchu (TW); Thomas Y.S. Shen, Hsinchu (TW)

(73) Assignee: APEX BIOTECHNOLOGY CORP.

(21) Appl. No.: 13/407,247

(22) Filed: Feb. 28, 2012

Related U.S. Application Data
(62) Division of application No. 12/397,394, filed on Mar. 4, 2009.

(30) Foreign Application Priority Data
Jun. 25, 2008 (TW) 097123784

(51) Int. Cl. G01N 33/72

(52) U.S. Cl. 205/777.5

(57) ABSTRACT

A hemoglobin-detecting electrode test strip is provided, which includes an insulating substrate; an electrode system; an insulating layer that partially covers the electrode system so that one uncovered portion on the electrode system forms an electrochemical reaction region and another uncovered portion on the electrode system forms a conductive wire connecting region; and a reaction layer including an electron mediator and an anion surfactant and at least partially covering the electrochemical reaction region. A device for detecting hemoglobin is further provided, which includes the hemoglobin-detecting electrode test strip and an electrochemical sensor.

### RELATED APPLICATION INFORMATION

This application is a divisional copending application Ser. No. 12/397,394 filed Mar. 4, 2009, and incorporates the same by reference.

### FIELD OF THE INVENTION

The present invention relates to an electrochemical electrode test strip, and particularly to a hemoglobin-detecting electrode test strip which is easy in operation and accurate in measurement and can be used for home monitoring and blood bank screening, and a device comprising the same.

### DESCRIPTION OF THE PRIOR ART

Hemoglobin is also referred to as hematein. Hemoglobin in the blood will bind with oxygen to form oxyhemoglobin, which will decompose into hemoglobin and oxygen when the blood is transported to a cell with low oxygen content in human body, whereby oxygen will be used by the cell and hemoglobin will be transported back to the lung with the blood to bind with oxygen again; in this way, hemoglobin is repeatedly circulated and thus fulfills the task of oxygen transportation in the body. The Food and Drug Administration (FDA) prescribes that the minimum standard value for blood donation of hemoglobin is 12.5 g/dl. A much lower hemoglobin level in the blood indicates anaemia, and a much higher hemoglobin level indicates polycythemia.

Generally, a hemoglobin detection system is required in a blood donation center for both blood screening and protection for blood donors. There are numerous detecting methods, such as chemical method, gas testing method, specific gravity method, and colorimetric method, and the two most commonly used methods at present are cupric sulfate falling drop method and hemoglobin analyzer (for example, HemoCue manufactured by HemoCue Inc.). The cupric sulfate falling drop method is used to measure the specific gravity of whole blood and not hemoglobin. However, this method suffers from many interfering factors, for example, the specific gravity of whole blood will be increased with high plasma protein concentrations, thereby resulting in detection error; furthermore, the spent liquor after detection may cause biological contamination. The HemoCue is a small instrument and only requires a short time for detection. However, it is based on optical detection principle and thus is expensive, greatly limiting the use thereof in general home care.

With the advance in medical science and the current requirements for health care, point-of-care testing (POCT) products are increasingly popular. The trend of development of detection systems is towards low cost, high detection speed, small size, and convenience for use. Among home detection systems, those based on electrochemical principle are relatively cheaper in general.

In view of the disadvantages of the known hemoglobin detecting systems described above, there is a need for an electrochemical detection system which is inexpensive, easy in operation, accurate in measurement, simple in preparation, and suitable for home monitoring and blood bank screening.

### SUMMARY OF THE INVENTION

One object of the invention is to provide a hemoglobin-detecting electrode test strip which includes an insulating substrate; an electrode system having a conductive film, in which the conductive film is coated onto a surface of the insulating substrate to form a working electrode and a reference electrode which are separated and not in contact with each other; an insulating layer that partially covers the electrode system so that one uncovered portion on the electrode system forms an electrochemical reaction region and another uncovered portion on the electrode system forms a conductive wire connecting region; and a reaction layer at least partially covering the electrochemical reaction region and including an electron mediator and an anion surfactant.

Another object of the invention is to provide a device for detecting hemoglobin which includes the above-mentioned hemoglobin-detecting electrode test strip and an electrochemical sensor. The electrochemical sensor includes a microprocessor, a display, a connector, and an internal memory, in which the microprocessor calculates an electrical signal transmitted from the hemoglobin-detecting electrode test strip via the connector to obtain a hemoglobin content in a sample in the electrochemical reaction region, and a calculation result is shown on the display.

### BRIEF DESCRIPTION OF THE DRAWINGS

FIG. 1 shows a schematic drawing of the hemoglobin-detecting electrode test strip according to an embodiment of the present invention; and

FIG. 2 shows a schematic drawing of the device for detecting hemoglobin according to an embodiment of the present invention.

### DETAILED DESCRIPTION

“Insulating substrate” as used herein refers to a thin sheet having a flat surface and insulating property. The material of the insulating substrate can be any material known in the art that is suitable as the insulating substrate of the electrochemical electrode test strip, including, but not limited to, polyvinyl chloride (PVC), glass fiber (FR-4), polyether sulfone (PES), bakelite, polyethylene terephthalate (PET), polycarbonate, glass, or ceramic (CEM-1).

“Electrode system” as used herein refers to an electrode comprising at least two conductive films (for use as a working electrode and a reference electrode, respectively) which are separated and not in contact with each other. The material of the conductive films can be any material known in the art that is suitable for the electrode system of the electrochemical electrode test strip. Preferably, the conductive films are coated by screen printing metal films (i.e., screen-printing electrodes, as disclosed in U.S. Pat. No. 6,923,894 B2) or by adhering metal films (as disclosed in U.S. Pat. No. 6,254,736 B1) onto a surface of the insulating substrate. Suitable materials for the metal films include, but are not limited to, gold, silver, platinum, or palladium, and suitable printing inks for the screen printing include, but are not limited to, carbon ink, gold ink, silver ink, a mixture of carbon ink and silver ink, volatile graphite, copper ink, or a combination thereof, for example, printing with silver ink followed by carbon ink. In an embodiment of the present invention, the screen-printing electrode includes a silver ink layer and a carbon ink layer covering the silver ink layer. In an embodiment of the present invention, the electrode system is partially covered with the insulating layer, so that the working electrode and the reference electrode not covered by the insulating layer at one end form an electrochemical reaction region and another uncovered portion at the other end forms a conductive wire connecting region. The conductive wire connecting region is used to connect the hemoglobin-detecting electrode test strip with an electrochemical sensor, and transmits an electrical effect induced by a detected sample during the electrochemical reaction.

“Insulating layer” as used herein refers to a layer formed from a material having insulating property and partially covering the electrode system. The material of the insulating layer can be any material known in the art that is suitable as the insulating layer of the electrochemical electrode test strip, including, but not limited to, polyether sulfone, polypropylene (PP), polyvinyl chloride, or polyvinyl alcohol (PVA). The insulting material is coated onto the electrode system through a screen printing process. In an embodiment of the present invention, the insulating layer is about 0.01 to about 0.6 mm in thickness.

“Reaction layer” as used herein refers to a thin layer at least partly covering the electrochemical reaction region, which can be formed by dripping a reaction layer formulation capable of reacting with a detected sample onto the electrochemical reaction region. In an embodiment of the present invention, the reaction layer formulation includes an electron mediator and an anion surfactant.

“Electron mediator” as used herein refers to a substance which can be changed from oxidation state to reduction state after reacting with a sample to be detected in whole blood. When in reduction state, the electron mediator can be inversely reacted to return to oxidation state by applying an external voltage to the test strip, at which point, the potential, resistance, or current changes of the chemical reaction can be transmitted to the terminal at the other end of the electrode system via the working electrode and the reference electrode capable of contacting with the reaction layer. Suitable electron mediators in the present invention include, but are not limited to, potassium ferricyanide, flavin adenine dinucleotide (FAD), nicotinamide, or nicotinamide adenine dinucleotide (NADH). The content of the electron mediator is about 3% to about 20%, preferably about 3% to about 15%, and more preferably about 3% to about 9% (by weight) of the reaction layer formulation. In an embodiment of the present invention, the electron mediator is potassium ferricyanide, and the content thereof is about 3% to about 9% (by weight) of the reaction layer formulation.

“Anion surfactant” as used herein refers to a substance used to enhance the reaction of hemoglobin with the electron mediator, including, but not limited to, sodium dodecyl sulfate, sodium dodecyl sulfonate, desoxycholate, or a derivative thereof. In an embodiment of the present invention, the anion surfactant is desoxycholate or a derivative thereof, and the content thereof is about 0.5% to about 4%, preferably about 1.5% to about 4%, and more preferably about 1.5% to about 2.5% (by weight) of the reaction layer formulation. In another embodiment of the present invention, the anion surfactant is sodium dodecyl sulfate or a derivative thereof, and the content thereof is about 0.5% to about 2%, preferably about 0.5% to about 1%, and more preferably about 0.5% (by weight) of the reaction layer formulation. In a further embodiment of the present invention, the anion surfactant is sodium dodecyl sulfonate or a derivative thereof, and the content thereof is about 0.5% to about 2%, preferably 1% to about 2%, and more preferably about 1% (by weight) of the reaction layer formulation.

In an embodiment of the present invention, the reaction layer formulation further includes a water soluble polymer carrier. The water soluble polymer carrier is a substance which can cause the electron mediator to be attached to the insulating substrate after drying, and strengthen the contact between the reaction layer and a detected liquid sample. Preferably, the water soluble polymer carrier has a particle size of less than about 100 μm, so that it can be uniformly dispersed in the reaction layer formulation without precipitation. Suitable water soluble polymer carriers in the present invention can be any one known in the art which is useful in the electrochemical electrode test strip, including, but not limited to, polyvinyl alcohol (PVA), polyvinyl pyrrolidone (PVP), polyethylene glycol (PEG), gelatin, carboxymethyl cellulose, methyl cellulose, or a mixture thereof. In an embodiment of the present invention, the content of the water soluble polymer carrier is about 0.5% to about 10%, preferably about 0.5% to about 4%, and more preferably about 0.5% to about 2% (by weight) of the reaction layer formulation.

In the present invention, the hemoglobin-detecting electrode test strip can further include a hydrophilic net layer or a hydrophilic top or both at least fully covering the reaction layer, which are used to accelerate the absorption of the detected sample, so that the detected sample can quickly covers the electrochemical reaction region, to reduce the chance of detection error resulting from insufficient sampling caused by the non-uniform absorption of the detected sample. Suitable materials for the hydrophilic net layer in the present invention can be any material known in the art which is useful in the electrochemical electrode test strip, including, but not limited to, polyether sulfone, nylon, polyethylene (PE), or glass fiber. Suitable materials for the hydrophilic top cover in the present invention can be any material known in the art which is useful in the electrochemical electrode test strip, including, but not limited to, polyether sulfone, polypropylene, polyvinyl chloride, or polyvinyl alcohol.

The present invention further provides a device for detecting hemoglobin which includes the hemoglobin-detecting electrode test strip mentioned above and an electrochemical sensor.

In an embodiment of the present invention, the electrochemical sensor includes a microprocessor, a display, a connector, and an internal memory. The connector is an element used for connecting the hemoglobin-detecting electrode test strip and the electrochemical sensor to transmit an electrical signal generated from the electrochemical reaction of the reaction layer and the detected sample in the electrochemical reaction region to the microprocessor. The microprocessor can calculate the electrical signal received to obtain a hemoglobin content in the detected sample in the electrochemical reaction region. The internal memory is used to store correction parameters, measuring reaction time parameters, or a plurality of detection modes, which are for use in calculation by the microprocessor. The display is used to show the hemoglobin content calculated by the microprocessor.

In an embodiment of the present invention, the electrochemical sensor can further include an external memory, which is used to store correction parameters or measuring reaction time parameters that are inputted into the internal memory of the electrochemical sensor depending on the nature of the hemoglobin-detecting electrode test strip. In a preferred embodiment of the present invention, the internal and external memories are electrically erasable programmable read only memory (EEPROM).

In the present invention, the measuring reaction time parameters stored in the internal or external memories include a preceding reaction time and an electrochemical reaction time, in which the preceding reaction time is the time of the sample placing in the reaction layer, and the electrochemical reaction time is the time during which an voltage is applied. In an embodiment of the present invention, the preceding reaction time is less than about 40 s, preferably about 5 to 40 s, and more preferably about 20 s, and the electrochemical reaction time is about 5 to 20 s, preferably about 5 to 10 s, and more preferably about 5 s.

In order to make the content of the present invention more comprehensible, the embodiments of the present invention are described below with reference to the accompanying drawings. However, these embodiments are used to illustrate the present invention only, not limit the scope of the present invention.

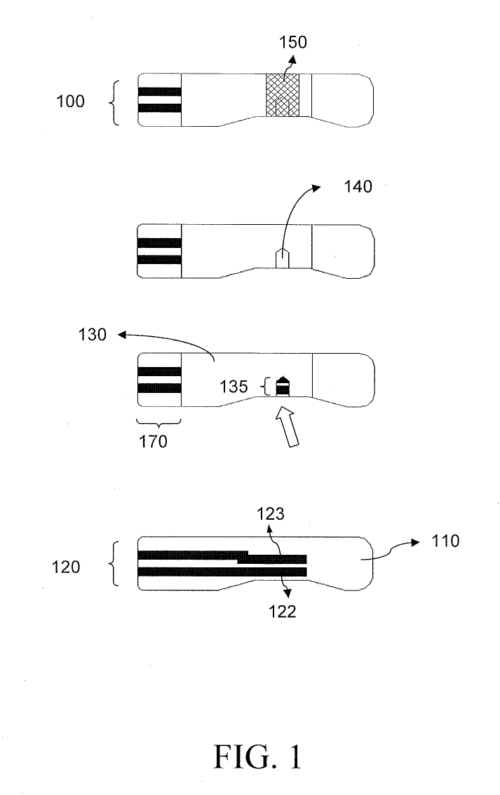

FIG. 1 shows a schematic diagram showing the hemoglobin-detecting electrode test strip according to an embodiment of the present invention. As shown in FIG. 1, an hemoglobin-detecting electrode test strip 100 includes an insulating substrate 110, an electrode system 120, an insulating layer 130, a reaction layer 140, and a hydrophilic net layer 150.

The electrode system 120 at least includes a working electrode 122 and a reference electrode 123, which are coated onto the insulating substrate 110 without contacting each other. The insulating layer 130 partially covers the electrode system 120, so that one uncovered portion on the electrode system forms an electrochemical reaction region 135 and another uncovered portion on the electrode system forms a conductive wire connecting region 170, in which a portion of the insulating layer 130 peripheral to the electrochemical reaction region 135 is partly removed to form a sampling end (as shown at the arrow in FIG. 1). The reaction layer 140 is covered on the electrochemical reaction region 135, and the hydrophilic net layer 150 at least fully covers the reaction layer 140.

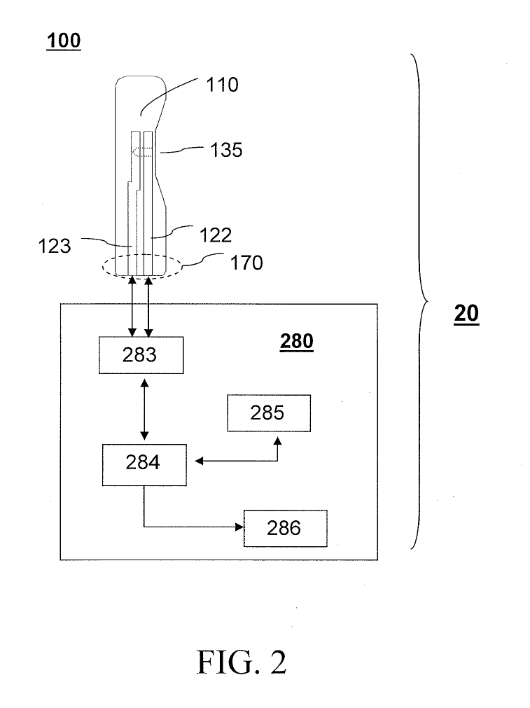

FIG. 2 shows a schematic diagram of the device for detecting hemoglobin according to an embodiment of the present invention. As shown in FIG. 2, a hemoglobin measuring system 20 includes the hemoglobin-detecting electrode test strip 100 and an electrochemical sensor 280.

The electrochemical sensor 280 includes a connector 283, a microprocessor 284, a display 286, and an internal memory 285. The microprocessor 284 in the electrochemical sensor 280 is electrically coupled to the conductive wire connecting region 170 of the hemoglobin-detecting electrode test strip 100 by the connector 283, thereby calculating an electrical signal transmitted form the hemoglobin-detecting electrode test strip via the connector to obtain a hemoglobin content in a detected sample in the electrochemical reaction region 135, and the calculation result will be shown on the display 286.

Furthermore, the electrochemical sensor 280 includes an internal memory 285, which can have a plurality of correction parameters, measuring reaction time parameters, or detection modes built in, and a plurality of other correction parameters, measuring reaction time parameters, or detection modes can be inputted from the exterior by an external memory (not shown) depending on the nature of the hemoglobin-detecting electrode test strip 100.

The following embodiments are used to further describe the present invention, and not limit the scope of the present invention. All modifications and variations which can be easily made by any persons of skill in the art fall within the disclosure of this specification and the scope of the appended claims.

### EXAMPLES

### Example 1

### Preparation of Hemoglobin-Detecting Electrode Test Stripe of the Present Invention

With the method disclosed in example 1 of U.S. Pat. No. 6,923,894 B2, a polymeric resin conductive carbon paste containing polyvinyl chloride and polyurethane was screen printed onto a flat surface of a PVC insulating substrate to form an electrode system composed of a working electrode and a reference electrode which were separated from each other. The substrate was dried. Then, an insulating layer was coated on the same surface of the substrate printed with the electrode system by keeping the working electrode and the reference electrode partially exposed to form a conductive wire connecting region and an electrochemical reaction region. Then, the substrate was dried.

Next, the following reaction layer formulation was dripped onto the electrochemical reaction region in a manner of constant volume and dried, to form a reaction layer.

The Reaction Layer Formulation Using Desoxycholate (DC)

| | |
|---|---|
| Water | 92.5% |
| potassium ferricyanide | 6.45% |
| desoxycholate | 1.05% |

The Reaction Layer Formulation Using Sodium Dodecyl Sulfate

| | |
|---|---|
| Water | 93.5% |
| potassium ferricyanide | 6.45% |
| sodium dodecyl sulfate | 0.05% |

The Reaction Layer Formulation Using Sodium Dodecyl Sulfonate

| | |
|---|---|
| Water | 92.55% |
| potassium ferricyanide | 6.45% |
| sodium dodecyl sulfonate | 1.0% |

Then, a PES hydrophilic net layer was directly placed on the insulating layer in such a manner that it at least fully covering the reaction layer, to produce a hemoglobin-detecting electrode test strip.

### Example 2

### Comparative Example

The hemoglobin-detecting electrode test strip of Example 1 produced with 0.5% of sodium dodecyl sulfate, 1% of sodium dodecyl sulfonate, or 1.5% of desoxycholate as the anion surfactant, and a conventional hemoglobin-detecting electrode test strip produced with a neutral surfactant (4.0% Triton X-100) were used to detect and analyze the hemoglobin concentration in whole blood with the same detection equipment. The detection results are shown in Table 1.

TABLE 1

| Preceding | mCV % | | | |
|---|---|---|---|---|
| Reaction | Triton X-100 | DC | Sodium dodecyl | Sodium dodecyl |
| Time | (4.0%) | (1.5%) | sulfate (0.5%) | sulfonate (1%) |
| 5 s | 7.70 | 4.58 | 8.06 | 7.94 |
| 10 s | 6.83 | 5.34 | 7.89 | 6.50 |
| 15 s | NA | 3.46 | NA | NA |
| 20 s | 8.23 | 3.32 | 7.49 | 6.19 |
| 25 s | 7.80 | 5.32 | 7.31 | 5.30 |
| 30 s | 8.61 | 4.80 | 8.20 | 7.90 |
| 40 s | 10.93 | 6.30 | 7.30 | 8.50 |

It can be seen from Table 1 that with the same reaction time, for example, when the preceding reaction time was 20 s, the use of the anion surfactants disclosed in the present invention obtains a mean coefficient of variation (mCV %) of 3.32, 7.49, and 6.19, respectively, all of which were lower than that of the use of the conventional neutral surfactant, 8.23, proving that the hemoglobin-detecting electrode test strip of the present invention was more accurate than the conventional electrode test strip.

Furthermore, as shown in Table 1, in the present invention, the mean coefficients of variation measured at different preceding reaction times, 5 s, 10 s, 15 s, 20 s, 25 s, 30 s, and 40 s were 4.58, 5.34, 3.46, 3.32, 5.32, 4.80, and 6.30, respectively, when the concentration of desoxycholate was 1.5%. It follows that in the present invention, the preceding reaction time is preferably about 10 to 30 s in order to prepare a hemoglobin-detecting electrode test strip with high accuracy.

### Example 3

### Efficacy of the Hemoglobin-Detecting Electrode Test Strip of the Present Invention

Table 2 shows the coefficients of variation of the electrode test strips prepared by using different desoxycholate concentrations according to the present invention. At the preceding reaction time was 20 s, tests were performed with hemoglobin detection samples of different concentrations of 12 g/dl, 15 g/dl, and 18 g/dl by using test strips containing 0.5%, 1.0%, 1.5%, 2.0%, 2.5%, and 4.0% of desoxycholate. The results are shown in Table 2.

TABLE 2

| | CV % | | | | | |
|---|---|---|---|---|---|---|
| Hemoglobin | DC | DC | DC | DC | DC | DC |
| Detection Sample | 0.5% | 1.0% | 1.5% | 2.0% | 2.5% | 4.0% |
| 12 g/dl | 8.3 | 9.5 | 4.3 | 5.3 | 5.2 | 6.1 |
| 15 g/dl | 6.2 | 5.1 | 4.2 | 4.1 | 4.2 | 5.3 |
| 18 g/dl | 6.4 | 4.2 | 4.9 | 5.1 | 4.5 | 4.4 |
| mCV % | 7 | 6.3 | 4.5 | 4.8 | 4.6 | 5.2 |

It can be seen from Table 2 that all the coefficients of variation of the hemoglobin-detecting electrode test strip containing 0.5% to 4.0% of desoxycholate were lower than that of the conventional test stripe, 8.23, except when 0.5% and 1.0% of desoxycholate were used to measure the hemoglobin detection sample of 12 g/dl. It follows that in the present invention, the content of desoxycholate used is preferably 1.5% to 4% and most preferably 1.5% to 2.5% in order to produce a hemoglobin-detecting electrode test strip with high accuracy.

In summary, the hemoglobin-detecting electrode test strip and the device including the same of the present invention are not only simple to manufacture, inexpensive, and easy in operation, but also can effectively improve the measurement accuracy of a conventional hemoglobin-detecting electrode test strip, and is suitable for home monitoring and blood bank screening.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

We claim:

1. A method for detecting hemoglobin in a detecting sample, comprising the steps of:
providing a device for detecting hemoglobin, which comprises an electrochemical sensor and a hemoglobin-detecting electrode test strip, wherein the electrochemical sensor comprises a microprocessor and a connector; and the hemoglobin-detecting electrode test strip comprises:
an insulating substrate;
an electrode system having a conductive film, wherein the conductive film is coated onto a surface of the insulating substrate to form a working electrode and a reference electrode which are separated and not in contact with each other;
an insulating layer that partially covers the electrode system, so that one uncovered portion on the electrode system forms an electrochemical reaction region and another uncovered portion on the electrode system forms a conductive wire connecting region; and
a reaction layer at least partially covering the electrochemical reaction region and comprising an electron mediator and an anion surfactant wherein the anion surfactant is desoxycholate or a derivative thereof, and the content of desoxycholate is 1.5 to 2.5% by weight of a formulation of the reaction layer; and
applying the detecting sample to the device, when the hemoglobin in the sample contacts the electrochemical reaction region of the hemoglobin-detecting electrode test strip, an electrical signal would generate and transmit to the microprocessor via the connector of the electrochemical sensor to obtain the hemoglobin content in the detecting sample.

2. The method according to claim 1, wherein the electron mediator is selected from the group consisting of potassium ferricyanide, flavin adenine dinucleotide (FAD), nicotinamide, and nicotinamide adenine dinucleotide (NADH).

3. The method according to claim 1, wherein the content of the electron mediator is about 3% to about 20% by weight of the reaction layer formulation.

4. The method according to claim 1, wherein the reaction layer further comprises a water soluble polymer carrier.

5. The method according to claim 4, wherein the water soluble polymer support is selected from the group consisting of polyvinyl alcohol (PVA), polyvinyl pyrrolidone (PVP), polyethylene glycol (PEG), gelatin, carboxymethyl cellulose, methyl cellulose, and a mixture thereof.

6. The method according to claim 4, wherein the content of the water soluble polymer carrier is about 0.5% to about 10% by weight of a reaction layer formulation.

7. The method according to claim 1, wherein the hemoglobin-detecting electrode test strip further comprises a hydrophilic net layer or a hydrophilic top at least fully covering the reaction layer.

8. The method according to claim 1, wherein the electrochemical sensor comprises a microprocessor, a display, a connector, and an internal memory.

9. The method according to claim 8, wherein the microprocessor calculates an electrical signal transmitted from the hemoglobin-detecting electrode test strip via the connector to obtain a hemoglobin content in a detected sample in the electrochemical reaction region, and a calculation result is shown on the display.

10. The method according to claim 8, wherein the internal memory is used to store correction parameters or measuring reaction time parameters.

11. The method according to claim 10, wherein the measuring reaction time parameters comprise a preceding reaction time and an electrochemical reaction time.

12. The method according to claim 11, wherein the preceding reaction time is less than about 25 s.

13. The method according to claim 12, wherein the preceding reaction time is about 5 to 20 s.

14. The method according to claim 13, wherein the preceding reaction time is about 20 s.

15. The method according to claim 11, wherein the electrochemical reaction time is about 5 to 20 s.

16. The method according to claim 15, wherein the electrochemical reaction time is about 5 to 10 s.

17. The method according to claim 16, wherein the electrochemical reaction time is about 5 s.

18. The method according to claim 1, wherein the electrochemical sensor further comprises an external memory for storing correction parameters that are transmitted into the electrochemical sensor.

19. The method according to claim 18, wherein the external memory is an electrically erasable programmable read only memory (EEPROM).
